(19) United States
(12) Patent Application Publication
(10) Pub. No.: US 2026/0355464 A1
(43) Pub. Date: Nov. 12, 2026

(54) ROLLING MEMBRANE CATHETER

(71) Applicant: BIOTRONIK AG, Buelach (CH)

(72) Inventors: Jeremy Wernli, Wettingen (CH); Matthias Wesselmann, Ruedlingen (CH); Azadeh Mehrabi, Meilen (CH)

(21) Appl. No.: 19/123,884

(22) PCT Filed: Dec. 4, 2023
(86) PCT No.: PCT/EP2023/084109

§ 371 Date: Apr. 24, 2025

(30) Foreign Application Priority Data
Dec. 12, 2022 (EP) 22212727.6

(51) Int. Cl. A61M 25/01; A61M 25/09

(52) U.S. Cl. CPC A61M 25/0119; A61M 25/0113; A61M 25/0122; A61M 2025/09125; A61M 2205/0216

(57) ABSTRACT

A catheter includes a rolling membrane and a guidewire. At least one decoupler is configured to decouple a motion of the rolling membrane from a motion of the guidewire or at least one motion compensator is configured to limit and compensate the motion of a guidewire advancement.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/084109, which was filed Dec. 4, 2023, which application claimed priority from EP Application 22212727.6, which was filed Dec. 12, 2022.

### FIELD OF THE INVENTION

A field of the invention concerns catheter devices and devices to treat stenosis.

### BACKGROUND

Medical treatment of a stenosis via percutaneous coronary intervention (PCI) can involve the insertion of a catheter into a blood vessel at the position of the stenosis. Rolling membrane catheters can allow catheter insertion into narrow cavities by everting a rolling membrane during a roll-out process. Typically, in a first step, a guidewire is introduced into the blood vessel and guided to the treatment site, e.g., the location of a stenosis. The rolling membrane of the rolling membrane catheter can then be everted, e.g., by providing pressure to the inside of the rolling membrane by a fluid unit connected to the catheter via a fluid inlet and moving one end of the rolling membrane (e.g. a proximal and/or inner end) distally. This can cause an eversion of the rolling membrane along the vessel walls, for example, without substantive friction. The one end of the rolling membrane can be attached to, e.g., an inner shaft which moves distally when the rolling membrane, attached to a distal end of the inner shaft, is everted. This everts the rolling membrane in a distal direction towards and possibly past a stenosis. The as-inserted rolling membrane can, for example, widen the stenosis or works as a guide catheter for other devices to be introduced to the blood vessel at the location of the stenosis.

However, there are a few issues that require care when using rolling membrane catheters. First, in conventional rolling membrane catheters, users can retract the rolling membrane when in an insufficiently inflated state such that the rolling membrane is clinched. Second, if used with a guidewire, the hydraulic pressure in the rolling membrane, when in an inflated state, typically clamps the guidewire and thus couples it with its motion. Due to the everting motion of the rolling membrane, the guidewire advances twice as fast as the leading edge of the everting rolling membrane. This requires particular care to control the advancement of the guidewire such as to avoid the risk to perforate the cardiovascular system of the patient. Further, the eversion and/or retraction of the rolling membrane is to be accompanied by a certain pressure applied to the rolling membrane. If a user, e.g., a health professional introducing the catheter, sets a pressure that is too high, the burst pressure of the rolling membrane can be exceeded, such that the rolling membrane might burst within the cardiovascular system of the patient.

### SUMMARY OF THE INVENTION

A catheter includes a rolling membrane and a guidewire. At least one decoupler is configured to decouple a motion of the rolling membrane from a motion of the guidewire or at least one motion compensator is configured to limit and compensate the motion of a guidewire advancement.

### BRIEF DESCRIPTION OF THE DRAWINGS

FIG. 1A Schematic representation of a rolling membrane catheter with coupled rolling membrane and guidewire motion and with the rolling membrane in a partly everted state.

FIG. 1B Schematic representation of a rolling membrane catheter with coupled rolling membrane and guidewire motion and with the rolling membrane in a further everted state with the leading edge of the rolling membrane advanced by a distance x and the inner member and the guidewire advanced by a distance 2x.

FIG. 2A Schematic representation of an outer shaft with an integrated locking structure in the unlocking position.

FIG. 2B Schematic representation of an outer shaft with an integrated locking structure in the locking position.

FIG. 3A Schematic representation of a rolling membrane catheter with decoupled rolling membrane and guidewire motion via a mechanical clamp and with the rolling membrane in a partly everted state.

FIG. 3B Schematic representation of a rolling membrane catheter with decoupled Un 5 rolling membrane and guidewire motion via a mechanical clamp and with the rolling membrane in a further everted state with the leading edge of the rolling membrane advanced by a distance x and a locked guidewire.

FIG. 4A Schematic representation of a rolling membrane catheter with decoupled rolling membrane and guidewire motion via a pressure-controlled clamp and with the rolling membrane in a partly everted state.

FIG. 4B Schematic representation of a rolling membrane catheter with decoupled rolling membrane and guidewire motion via a pressure-controlled clamp and with the rolling membrane in a further everted state with the leading edge of the rolling membrane advanced by a distance x and a locked guidewire.

FIG. 5A Schematic representation of a rolling membrane catheter with decoupled rolling membrane and guidewire motion via a pressure-controlled clamp and with the rolling membrane in a partly everted state with a fluid pressure provided to the volume between the rolling membrane and the guidewire.

FIG. 5B Schematic representation of a rolling membrane catheter with decoupled rolling membrane and guidewire motion via a pressure-controlled clamp and with the rolling membrane in a further everted state with the leading edge of the rolling membrane advanced by a distance x and a locked guidewire with a fluid pressure provided to the volume between the rolling membrane and the guidewire.

FIG. 6A Schematic representation of the retraction of a pressurized rolling membrane,

FIG. 6B Schematic representation of the retraction of a non-pressurized rolling membrane,

FIG. 7A to 7H Schematic representation of a rolling membrane catheter comprising a motion compensator,

FIG. 8A/8B Schematic representation of a rolling membrane catheter comprising a motion compensator with an automatic pressure reduction,

FIG. 9A/9B Schematic representation of another rolling membrane catheter comprising a motion compensator with an automatic pressure reduction.

### DETAILED DESCRIPTION OF THE PREFERRED EMBODIMENTS

The present invention relates to a catheter comprising a rolling membrane and various structures for facilitating practical use of such catheter. Further, corresponding systems comprising such catheter and at least one fluid unit are provided. The present invention further relates to the use of a tubular element comprising a friction-reducing element as a rolling membrane. It further relates to respective computer programs and methods.

According to a first aspect of the invention, a catheter comprising a rolling membrane can be provided. The catheter can further comprise at least one locking structure configured to lock the rolling membrane at least in part based on a fluid state of the catheter. For example, the rolling membrane can be locked, e.g. such that it cannot be everted or retracted, when no sufficient fluid pressure is provided to the rolling membrane. Then it can be advantageous to lock the rolling membrane and to prevent, e.g., the retraction of the rolling membrane for example by retracting a member attached to one end of the rolling membrane as this might clinch the rolling membrane. Thus, such locking can not only prevent damage to the catheter but further reduces risks for the patient associated with removing the damaged catheter. In some examples, the locking structure can additionally or alternatively be activated if the fluid pressure provided to the rolling membrane is too high. Hence, this can avoid that, e.g. by further retracting the rolling membrane, the membrane can burst.

In an example, locking can prevent an inner shaft of the catheter to move relative to an outer shaft of the catheter, wherein the rolling membrane can be connected to a distal end region of the inner shaft and a distal end region of the outer shaft. Thereby, the movement of the rolling membrane, i.e., the eversion and/or the retraction can be prevented.

The fluid state is to be understood as the state of a fluid within any volume of the catheter as described herein. The fluid state can relate, e.g., to a fluid pressure and/or a fluid flow. A fluid can be any liquid fluid, any gaseous fluid, and/or any combination thereof. The fluid state of the catheter can particularly relate to a fluid state of the membrane. For example, the locking structure can be configured to lock the rolling membrane as long as a fluid pressure applied to a rolling membrane volume is below a predetermined threshold, such as to prevent movement of the membrane when in a not sufficiently inflated state. The locking structure can be configured to release the rolling membrane when the fluid pressure applied to the rolling membrane volume reaches the predetermined threshold.

In some examples, the catheter comprises at least one locking structure that can be at least in part connected to at least a portion of the outer shaft of the catheter. This can for example be achieved in an advantageous configuration wherein the locking structure are an integral part of the outer shaft of the rolling membrane catheter, saving material and required space at the same time. This can improve the overall handling and safety.

In an example, one or more sections of the outer shaft can be formed by the locking structure, e.g., interlocking and/or friction-locking structure. To this end, a member can be provided on the outer shaft interacting with a member on an inner shaft to lock the motion of the rolling membrane. For example, the locking structure can further be shaped in such a way that interlocking and/or friction-locking is facilitated, e.g., by choosing rough and/or friction-increasing materials and/or by providing members and/or sites configured to interlock or otherwise engage with each other, like serrated sites. This can yield a compact, material-saving catheter design, reducing labor costs and material costs in production. The locking structure can for example comprise one or more ring-shaped elements replacing a section of the outer shaft, which are biased to engage with the inner shaft unless pressure provided to a lumen between inner and outer shaft lifts them off the inner shaft and thus unlocking the rolling membrane. However, additionally or alternatively, other elements, e.g. with a circular or any other cross section, like, e.g., a knob, integrated into the surface of the outer shaft, etc., can be employed. Several such non-ring-shaped elements can be distributed around a circumference of the outer shaft. The catheter can comprise at least one locking structure configured to be in a first configuration locking the motion of the rolling membrane when insufficient fluid pressure is provided to the catheter. Further, the locking structure can be configured to be in a second configuration unlocking the motion of the rolling membrane when sufficient fluid pressure is provided to the catheter. Herein, the term ‘locking’ can be understood as preventing a motion of at least one member relative to at least one other member of the catheter and ‘unlocking’ can be understood as allowing the motion of at least one member relative to at least one other member if the catheter. A member can, for example, be any structure/element/building block/(sub-)unit of the catheter. For example, when the locking structure is connected to the outer shaft, the locking structure can be in the second configuration when the locking structure are in a lifted position in which, e.g., there is no contact between the locking structure and the inner shaft and/or the respective counterpart connected to the inner shaft. The locking structure can further be in contact with the inner shaft and/or the respective counterpart when it is the first configuration. In some examples, the locking structure can be in constant contact with, e.g., the inner shaft but the force clamping the inner shaft, or any other member to be clamped for locking the rolling membrane, can be significantly different between the two configurations of the locking structure. Thereby, the locking structure provide a safety-mechanism preventing damaging the catheter and/or vessel walls. This can yield a user-friendly, mechanical feedback to a user operating with the catheter on whether the inner member should be moved or not which can improve safety.

In a further example, the catheter can comprise at least one locking structure comprising an elastic element configured to change its configuration based at least in part on the fluid state of the catheter. This can yield a low-cost locking structure functioning, e.g., without any electrical power which can optimize reliability and/or safety. Further, the elastic element can be particularly suitable to maximize the area of contact with the surface of the member to be contacted and locked. The elastic element can be for example be a silicone, rubber, and/or any other elastic element in a shape configured to change its configuration in response to a change in the fluid state, e.g., a changing fluid pressure. It can be pre-tensioned, for example. Such element can for example be at least one elastic tubular member and/or ring that clamps the inner shaft of the catheter when a low fluid pressure is provided to the volume between the outer shaft and the inner shaft, locking the inner shaft relative to the outer shaft and thus the rolling membrane. The elastic element can be provided to be under tension such that it clamps the inner shaft when no pressure or pressure below a predetermined threshold is provided. In an example, it can further be inflated at least partly, when a higher fluid pressure is provided to the volume between the outer shaft and the inner shaft such that it is lifted from the inner shaft (or pressure applied to the inner shaft is reduced), allowing for motion of the inner shaft relative to the outer shaft and thus the rolling membrane. In an example, the elastic element can be at least one knob configured to change its configuration such that it either locks or unlocks the rolling membrane depending on its fluid state-dependent configuration. In some examples, the elastic element can comprise at least one spring and/or spring-like element that can be configured to hold the locking element in the locking position unless sufficient fluid pressure is provided to the catheter counteracting the force of the spring and allowing for motion of the rolling membrane. In some examples, the elastic element can comprise an elastic layer with a finger-like element arranged to lock the inner shaft, unless sufficient pressure is applied to lift the elastic layer and thus the finger-like element from the inner shaft. Such an elastic layer can be robust, reliable, and safe solutions suitable to be integrated into a catheter.

In some examples, the catheter can further comprise a fluid state sensor. The at least one locking structure can be in the locked and/or unlocked configuration based at least in part on measuring the fluid state provided to the catheter by the fluid state sensor. The fluid sensor can also be provided to the catheter in addition to any locking mechanism described herein.

According to a second aspect of the invention, a catheter comprising a rolling membrane is provided which prevents unintentional advancement of guidewire (i.e. the unwanted advancement of the guidewire at about twice the speed of the leading edge of the rolling membrane). This can be done by using either at least one decoupler configured to decouple a motion of the rolling membrane from a motion of the guidewire or by using at least one motion compensator configured to limit and compensate the motion of a guidewire advancement. Such a catheter comprises a rolling membrane, a guidewire, and at least one decoupler configured to decouple a motion of the rolling membrane from a motion of the guidewire or at least one motion compensator configured to limit and compensate the motion of a guidewire advancement. Decoupling the motion of the rolling membrane and the motion of the guidewire can solve some of the problems related to unwanted guidewire advancement described herein at least in part. It can further increase safety and provide a user-friendly catheter. The guidewire can be used as a tool to guide the rolling membrane such that the guidewire can be introduced into, e.g., a blood vessel to the treatment site like a stenosis. The tubular rolling membrane can be everted with the guidewire to and beyond the treatment site as it can envelop the guidewire such that its eversion is determined by the positioning of the guidewire. It can be advantageous to always be able to move the guidewire and the rolling membrane independently from each other. For this purpose, their motion can at least partially decoupled.

The at least one decoupler can be any structure providing any mechanism capable of decoupling the motion of the guidewire and the rolling membrane. This decoupling can allow for motion of the rolling membrane when the guidewire is not moving and/or allow for motion of the guidewire when the rolling membrane is not moving. Further, the guidewire and the rolling membrane can move independently from each other simultaneously and/or consecutively, e.g., at different velocities, accelerations, and/or in different directions. The catheter can comprise at least one decoupler comprising a clamping element configured to restrict the movement of the guidewire, preferably in a longitudinal direction. Clamping the guidewire can be an effective decoupling mechanism that is suitable to overcome the clamping force of the rolling membrane onto the guidewire. Thereby, it can increase safety.

The clamping element can for example be a mechanical clamp clamping the guidewire via an interlocking (e.g. in a form-fitting manner) and/or a friction-locking connection between the clamping element and the guidewire. For example, the clamping element can clamp the guidewire via at least two stamps configured to clamp the guidewire, a locking screw, and/or in a notch. Alternatively, the clamping element can be an elastic element (e.g. a silicone or rubber) and/or any other elastic element in a shape configured to change its configuration in response to a change in the fluid state (e.g. a changing fluid pressure). The clamping element can be tightened to clamp the guidewire once or the clamping force can be adjusted to the clamping force required at that moment. In an example, the clamping can be controlled by the user and/or it can be controlled (semi-) automatically. The user can further be instructed to clamp the guidewire by the clamping element based at least on part on the fluid state and/or the motion state of the rolling membrane, e.g., as sensed by a respective sensor.

The catheter can in some examples comprise decoupler comprising at least one friction-reducing element on an outer side of the rolling membrane. This can have the advantage to reduce the friction between the rolling membrane and the guidewire. Thus, the guidewire can remain stationary while the rolling membrane is everted and/or retracted as a substantially weaker force can be applied by the rolling membrane onto the guidewire. This can further be advantageous as the rolling membrane can in this way be manipulated independently from any other member of the catheter.

The outer side of the rolling membrane is the side that is in contact with the vessel wall when the rolling membrane is fully everted and in contact with the guidewire when it is fully retracted.

In some examples, when the rolling membrane is in a (fully) retracted state and a fluid pressure is provided to the rolling membrane, the rolling membrane is inflated such that at least a part of the outer side of the rolling membrane can be brought into contact with at least a portion of the guidewire. The resulting clamping force onto the guidewire can depend on an area of contact and the fluid pressure provided to the rolling membrane. Due to the resulting clamping force and friction between the rolling membrane and the guidewire, the rolling membrane can drag the guidewire with it when it is everted or retracted. The force acting onto the guidewire generally depends on the area of contact, the normal force, i.e., the clamping force, and the coefficient of friction, which is an empirical property of the two materials in contact with each other. The friction-reducing element can reduce the coefficient of friction. This can either reduce the force acting onto the guidewire such that either the force dragging the guidewire with the rolling membrane is simply reduced and/or the force can be reduced so much that the guidewire remains stationary when the rolling membrane is everted and/or retracted.

For example, the friction-reducing element can comprise a hydrophilic portion. The friction-reducing element can further, for example, comprise a film on the whole outer side of the rolling membrane or it can only cover a part of the outer side rolling membrane. The friction-reducing element can further be an integral part of the rolling membrane such that, e.g., the full rolling membrane consists of the friction reducing element and/or sections of the rolling membrane comprise the friction-reducing element. In some examples, the friction reducing element can cover a part of the outer side of the rolling membrane in form of any pattern which can be, e.g., striped, dotted, or a chessboard-pattern. Such pattern might further, e.g., vary along the length in axial direction of the rolling membrane, for example to provide a spatially altering coefficient of friction, adjusted to the respective area of contact between the guidewire and the rolling membrane depending on the respective state of eversion. For example, the portion of the rolling membrane that is everted first can be provided with a friction-reducing element as the area of contact is large when that portion is in contact with the guidewire. The last portion of the rolling membrane to be everted can be only covered in part by the friction-reducing element or can not be provided with the friction-reducing element. This can be sufficient as the contact area is small when the rolling membrane is almost fully everted. Thus, the friction force can be too small already to drag the guidewire with the rolling membrane. Hence, at that stage no friction reduction can be required anymore to decouple the motion of the rolling membrane and the guidewire. Generally, such pattern can be adjusted in any way tailoring the interaction between rolling membrane and guidewire. The friction-reducing element can, in some examples comprise one or more polymers, liquid films, etc. The friction-reducing element can further be adapted to be permanently applied to the rolling membrane such that no parts detach from the rolling membrane and contaminate the treated lumen.

The at least one motion compensator can comprise a torque element (also denoted as guidewire torquer) and optionally an elastic object, preferably a spring (in which the load is an axial force) like a coil spring. This ensures that the guidewire advancement in the rolling membrane catheter is limited and (cyclically) compensated independently of the user, thereby increasing safety in a clinical setting. Optionally the motion compensator can be provided with automatic pressure reduction. Therefore, the at least one motion compensator can further comprise a sealing element, preferably a ball valve or a sliding seal.

The catheter can comprise a first fluid inlet configured to be connected to a first fluid unit. The first fluid inlet can be configured for pressurization of a pressure-controlled clamping element. The clamping element can be configured to clamp the guidewire when a fluid pressure is provided to the first fluid inlet by the first fluid unit. For example, the clamping element can be inflated by the fluid pressure, upon which it clamps the guidewire. A fluid-state-based control can be an advantageous control system compatible with the necessary flexibility of the catheter. Further, it can provide a clamping force clamping the guidewire (semi-)automatically and/or only when required, therefore increasing safety, and improving the catheter handling in a user-friendly way.

The clamping element can be a mechanical clamp as described herein. Such mechanical clamp can be activated to clamp the guidewire in a (semi-)automatic way, e.g., as instructed by a control unit based at least in part on a fluid state sensed by a respective sensor. In some examples, the clamping element can be configured to directly respond to pressure provided to it. For example, the clamping element can comprise an element configured to be inflated and/or otherwise change its configuration depending on the pressure provided to it, e.g., by the fluid unit via the first fluid inlet. For example, when pressure below a predetermined threshold is provided to the clamping element it can not clamp the guidewire and/or when a fluid pressure above a predetermined threshold is provided to it, it can clamp the guidewire. The clamping element can, e.g., comprise an (elastic) ring with an inner diameter placed around the guidewire. When pressure is provided to the outer side of the ring, the inner diameter can be reduced such that the guidewire is clamped, the guidewire cannot move, and the motion of the rolling membrane and the guidewire is thus decoupled. The guidewire can be released at any time by adjusting the pressure provided to the clamping element. Any of the functionalities described with respect to the clamping element or any other function described herein can be implemented as steps of a method and/or instructions of a computer program.

The catheter can comprise a second fluid inlet configured to be connected to the first fluid unit and/or another fluid unit. The second fluid inlet can be configured for pressurization of the rolling membrane. The first fluid unit can further be configured to determine the fluid state of the rolling membrane of the catheter. Using the same fluid unit to determine the fluid state of the rolling membrane and to control the clamping element can simplify the system, make it more user-friendly, reduce material costs, and result in a suitable adjustment of the clamping force to the fluid state of the rolling membrane.

In some examples, different fluid units can be connected to the first and second inlet to control the two respective fluid states separately, possibly in mutual adjustment for synchronized operation. The same fluid unit can be connected to the first fluid inlet and the second fluid inlet providing the same fluid state to both. In particular, the guidewire can thus automatically (not) be clamped when the membrane is (not) inflated. The first fluid inlet can connect the fluid unit with the clamping element, activated by at least one parameter associated with the fluid state as described herein. The second fluid inlet can connect the fluid unit with the space between the outer shaft and the inner shaft of the catheter limited by the rolling membrane at the distal end of the catheter and by a sealing structure at the proximal end of the catheter, which is referred to as the rolling membrane volume herein. The fluid state of the rolling membrane can be understood as the fluid state provided to the rolling membrane volume. In an example, the rolling membrane can be considered in an inflated state, when the fluid pressure is above a minimum value (and below a burst pressure), wherein with a fluid pressure below the minimum value the rolling membrane is considered as only partly inflated.

In some examples, the at least one decoupler or the at least one motion compensator of the catheter is at least in part arranged at a portion of the catheter proximal to the rolling membrane. This can yield the advantage that the decoupler can not interfere with the regular operation of the catheter, e.g., the insertion of the distal end of the catheter into the patient and/or the eversion of the rolling membrane. The decoupler as described herein can be mounted directly to, e.g., a proximal end portion of the outer shaft and/or the inner shaft. In some examples, the decoupler can be connected to the proximal end portion of the outer shaft and/or the inner shaft via a frame and/or a leg. The frame and/or a leg can extend from the proximal end region of the outer shaft further into the proximal direction to place the decoupler proximally beyond the proximal end of the outer shaft. The frame and/or leg can have the shape of a bracket providing space for a structure to control the motion of the inner shaft, e.g., via a handle at the proximal end of the inner shaft.

In some examples, the catheter can comprise a third fluid inlet configured to be connected to a second fluid unit. The second fluid unit can be configured to provide a fluid pressure to the volume between the rolling membrane and the guidewire. This second fluid pressure can have an advantageous effect as it counteracts the fluid pressure provided to the other side of the rolling membrane (the rolling membrane volume) as described herein: For example, the additional pressure provided by the second fluid unit can at least partly lift the rolling membrane off the guidewire when the two pressures are adjusted to each other accordingly (and/or at least reduce the clamping force between the rolling membrane and the guidewire). This can reduce the friction force between rolling membrane and guidewire and thus decouple the motion of the rolling membrane and the guidewire. This can prevent the unwanted advancement of the guidewire at twice the speed of the leading edge of the rolling membrane. The third fluid inlet can be configured for pressurization of the volume between the inner shaft of the rolling membrane. In an example, the third fluid inlet can be located at the inner shaft, e.g., at a proximal end region of the inner shaft near the handle. This way it can not interfere with the operation of the catheter as described herein. The second fluid unit, like any other fluid unit, can be an external or an integral part of a catheter system. It can be controlled by a health professional or in a (semi-)automatic way, e.g., it can be adjusted based at least in part on the fluid state of the rolling membrane volume. In an example, the fluid pressure provided to the volume between the rolling membrane and the guidewire can be adjusted to the fluid pressure provided to the rolling membrane volume such that only a small portion of the rolling membrane is in contact with the guidewire, e.g., only along a ring at the distal end of the rolling membrane. This can reduce the friction enough to decouple the motion of the rolling membrane and the guidewire without necessarily opening an inner lumen of the rolling membrane. The fluid pressure provided to the volume between the rolling membrane and the guidewire can be adjusted at least in part automatically based at least in part on the fluid pressure provided to the rolling membrane volume. This can, e.g., be realized by coupling the two respective fluid units for synchronized operation or even using one and the same fluid unit for the respective fluid inlets.

In one aspect, a system comprising a rolling membrane catheter as described herein and at least one fluid unit can be provided. The fluid unit can be configured to provide a fluid pressure to at least two of the following: the first fluid inlet, the second fluid inlet, and a third fluid inlet. Connecting one fluid unit to more than one inlet can provide a cost-effective and more user-friendly system. Further, it can be advantageous to provide the same fluid states to different elements as their activation as described herein can be inherently coupled in an advantageously adjusted way. Further, this configuration provides a high flexibility and the possibility to adjust the use of at least one fluid unit to the planned treatment and/or other patient-related factors. The at least one fluid unit can be connected to the respective inlets via flexible hoses or any other connection element. The at least one fluid unit can be controlled by a health professional or in an automatic and/or semi-automatic way.

According to a third aspect of the invention, a catheter comprising a rolling membrane is provided. The catheter can comprise at least one pressure release valve and/or at least one friction-reducing element on an inner side of the rolling membrane. In some examples, the at least one pressure release valve can open when the fluid pressure exceeds a predetermined threshold. Thus, bursting of the rolling membrane can be prevented in a safe and user-friendly way. The at least one pressure release valve can for example be located at a proximal end portion of the outer shaft.

In an example, the friction-reducing element on the inner side of the rolling membrane can be present in addition or alternatively to a friction-reducing element on the outer side of the rolling membrane. Further, the friction-reducing element on the inner side of the rolling membrane can have any properties described herein in reference to the friction-reducing element on the outer side of the rolling membrane. The friction-reducing element on the inner side of the rolling membrane can reduce friction between two portions of the rolling membrane in contact with each other, as, e.g., when the rolling membrane is clamped in a stenosis such that it is not in its fully inflated state. When the portions in contact with each other can move relative to each other without substantial friction (or at least reduced friction) as afforded by the friction-reducing element, the rolling membrane can for example be retracted in a rolling motion without clinching/folding. The friction between two portions of the rolling membrane in contact with each other can be high, for example when no friction-reducing element is available. In such examples, the retraction of one end of the rolling membrane, e.g., via the retraction of the inner shaft, can drag the two contacting portions of the layer with it. This is incompatible with the intended rolling motion of the rolling membrane. Rather, the rolling membrane can be compressed along the axial direction and folded. By of the friction-reducing element this issue can be avoided and a correct rolling-in of the membrane can be enabled even in difficult situations.

A fourth aspect of the invention is the use of a tubular element comprising a friction-reducing element applied to at least a part of an outer side of the tubular element and/or to at least a part of an inner side of the tubular element as a rolling membrane for a rolling membrane catheter. The tubular element used as a rolling membrane can have any feature described herein in relation to the rolling membrane, the friction-reducing elements, the catheter and/or any other members of the catheter.

According to a fifth aspect of the invention, a computer program comprising instructions can be provided, such that when the program is executed, it can cause a rolling membrane catheter to clamp a guidewire of the catheter by a clamping element to decouple a motion of a rolling membrane of the catheter from a motion of the guidewire. The computer program can comprise instructions implemented according to any feature described herein and further any such instruction of a computer program can be realized as a step of an according method. Such computer program can be advantageous for automatization, increasing reliability, reduce the risk of user errors and it can improve the over-all handling and/or performance of the respective catheter. Such computer program can in some examples be based at least in part on an artificial intelligence, AI, assistant as described herein. Such AI might suggest and/or determine at least one parameter associated with the operation of the catheter.

In a further example, a computer program comprising instructions can be provided, such that when the program is executed, it can cause the catheter (e.g. as described herein) to lock the rolling membrane at least in part based on a fluid state of the catheter. In some examples, it can cause the at least one locking structure of a rolling membrane catheter to be in a first configuration locking the rolling membrane when insufficient fluid pressure is provided to the catheter. Further, it can cause the at least one locking structure to be in a second configuration unlocking the motion of the rolling membrane when sufficient fluid pressure is provided to the catheter. This can for example involve signaling from a sensor sensing the fluid state of the rolling membrane volume, for example. Therein, the signaling can be based at least in part on at least one parameter associated with the fluid state of rolling membrane volume. This can bring the at least one locking structure into contact with the inner shaft, e.g., in case the at least one locking structure can be at least in part connected to at least a portion of an outer shaft of the catheter (as described herein). When the at least one locking structure comprises an elastic element, the instructions of the computer program, when executed, can cause the elastic element to change its configuration based at least in part on the fluid state of the catheter.

Herein, a distal direction and a proximal direction, whenever referred to, relates to a leftward and a rightward direction, respectively, in all figures described in the following.

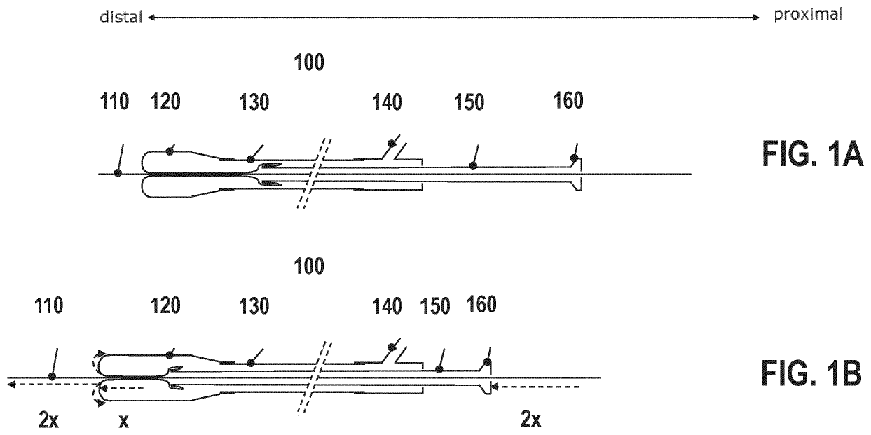

FIG. 1A shows a schematic representation of a rolling membrane catheter 100 with coupled rolling membrane 120 and guidewire 110 motion and with the rolling membrane 120 in a partly everted state. The catheter comprises an outer shaft 130 and inner shaft 150. A second fluid inlet 140 is provided and configured to be connected to a fluid unit (not shown) determining the fluid state of the rolling membrane 120 by providing, e.g., a fluid pressure to the rolling membrane volume limited by the rolling membrane 120, the outer shaft 130, and the inner shaft 150. The exemplary embodiment of FIG. 1A further comprises a handle 160 at the proximal end of the inner shaft 150. A health professional can move the inner shaft 150 relative to the outer shaft 130 by this handle 160. They can move the inner shaft 150 in the distal direction to evert the rolling membrane 120 and in the proximal direction to retract the rolling membrane 120. During both, eversion and retraction, the rolling membrane 120 is ideally in an inflated state such that it is everted and/or retracted in a rolling motion.

FIG. 1B shows the exemplary embodiment of the catheter 100 of FIG. 1A wherein the rolling membrane 120 is in a further everted state, via movement of the inner shaft 150 in the distal direction, as the leading edge of the rolling membrane 120 is advanced by a distance x. The inner shaft 150 is in a different position, dislocated by a distance 2x compared to the situation of FIG. 1A. The guidewire 110, which is clamped by the rolling membrane 120 is also dislocated by a distance 2x in the distal direction compared to its position shown in FIG. 1A. FIG. 1B thus shows an exemplary scenario for coupled motion of the guidewire 110 and the rolling membrane 120 bearing the risk of unwanted guidewire 110 advancement into, e.g., a blood vessel, one of the problems with rolling membrane catheters 100. Together, FIGS. 1A and 1B illustrate the basic working principle and the basic building blocks of a rolling membrane catheter 100.

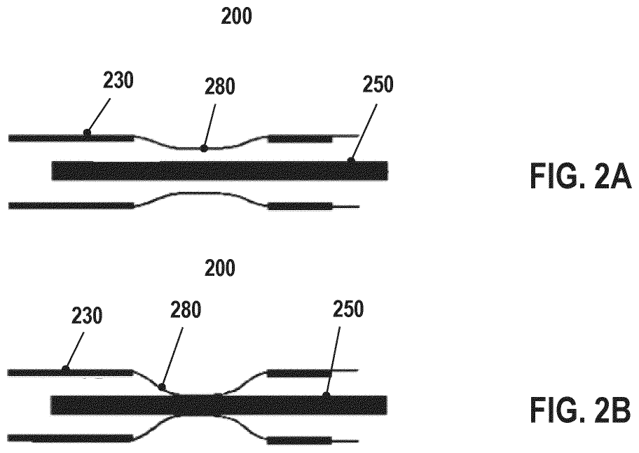

FIG. 2A shows a schematic representation of an outer shaft 230 of a catheter 200 with an exemplary locking structure 280 in the unlocking position. The exemplary locking structure 280 is integrated in the outer shaft 230. The locking structure 280 can comprise an elastic ring or tube. The locking structure 280 of the shown exemplary can comprise an elastic tubular element which is integrated as one portion into the tubular outer shaft 230. In some examples, the elastic tubular element can abut around an outer surface of the outer shaft 230. The unlocking position of the exemplary embodiment shown in FIG. 2A is such that the elastic ring of the locking structure is lifted off the inner shaft 250 due to sufficient pressure provided to the volume between the outer shaft 230 and the inner shaft 250. The section shown in FIG. 2A can only be a part of the full outer shaft 230 and there can be multiple such locking structure 280 comprised in the outer shaft 230. The multiple locking structure can be identical or different, e.g., differing in length in the axial direction, material, and/or shape.

FIG. 2B shows a schematic representation of the embodiment of FIG. 2A with an outer shaft 230 with an integrated locking structure 280 in the locking position. The locking structure 280 can be arranged around the outer shaft 230. The locking position of the exemplary embodiment shown in FIG. 2A is such that the elastic ring of the locking structure is not lifted off the inner shaft 250 due to insufficient pressure provided to the volume between the outer shaft 230 and the inner shaft 250. Therefore, the locking structure 180 clamps the inner shaft 250 and connects the outer shaft 230 and the inner shaft 250 such that they cannot move relative to each other. The locking structure 280 can comprise a form other than tubular. For example, the locking structure 280 can comprise at least one knob, piston, pin, and/or membrane. Any of these locking structure 280 can be configured to be in the unlocking position when, e.g., sufficient fluid pressure is provided to the catheter 200. In the unlocking position, the locking structure 280 are passive such that they do not lock any member of the catheter 200. Further, any locking structure 280 can be configured to change its configuration as described herein, e.g., when insufficient fluid pressure is provided to the catheter 200, into their active locking position locking at least one member of the catheter 200.

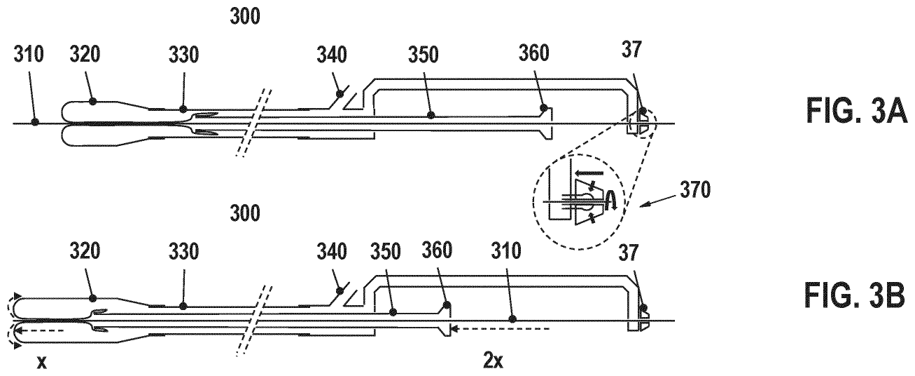

FIG. 3A shows a schematic representation of a rolling membrane catheter 300 with decoupled rolling membrane 320 and guidewire 310 motion via a clamping element 37 and with the rolling membrane in a partly everted state. The clamping element 37 can extend from a proximal end of the outer shaft 330 and/or from a proximal end of a handpiece at the proximal end of the outer shaft 330 further proximally. The clamping element 37 shown in FIG. 3A can be a mechanical clamp 370, wherein the guidewire 310 can be mechanically clamped via, e.g., a locking screw. Any other locking mechanism described herein is possible as well either alternatively or in addition to the shown locking screw. Further, the catheter 300 comprises the second fluid inlet 340 configured to be connected to a fluid unit (not shown) determining the fluid state of the rolling membrane 320. The catheter comprises further an inner shaft 350 and a handle 360.

FIG. 3B shows the exemplary embodiment of the catheter 300 of FIG. 3A wherein the rolling membrane 320 is in a further everted state as the leading edge of the rolling membrane 320 is advanced by a distance x. The inner shaft 350 is in a different position, dislocated, e.g. via the handle 360, by a distance 2x compared to the situation of FIG. 3A. The guidewire 310, which is clamped by the rolling membrane 320 is clamped by the clamping element 37 such that it is at the same position as in FIG. 3A, as the clamping force of the clamping element 37 exceeds the clamping force of the rolling membrane 320. FIG. 3B thus shows an exemplary scenario for decoupled motion of the guidewire 310 and the rolling membrane 320 reducing the risk of unwanted guidewire 310 advancement into, e.g., a blood vessel.

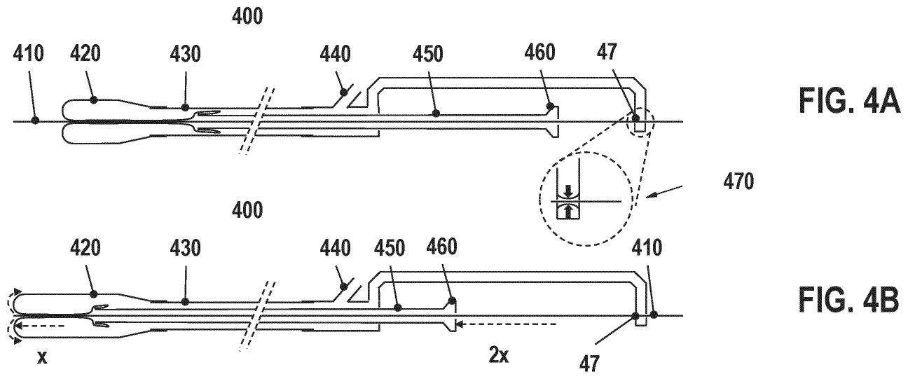

FIG. 4A, in analogy to FIG. 3A, shows a schematic representation of a rolling membrane catheter 400 with decoupled rolling membrane 420 and guidewire 410 motion via a clamping element 47 and with the rolling membrane in a partly everted state. The clamping element 47 shown in FIG. 4A is a pressure-controlled clamp 470, wherein the guidewire 410 is clamped via an elastic member brought into contact with the guidewire 410 to clamp the guidewire 410 when sufficient fluid pressure is provided to the clamp 470. The clamping element 47 can be arranged similarly with respect to outer shaft 430 as outlined with reference to clamping element 37 and outer shaft 330 of FIGS. 3A and 3B. Further, the catheter 400 comprises the second fluid inlet 440 configured to be connected to a fluid unit determining the fluid state of the rolling membrane 420. The catheter comprises further an inner shaft 450 and a handle 460.

Analogously to FIG. 3B, FIG. 4B shows the exemplary embodiment of the catheter 400 of FIG. 4A wherein the rolling membrane 420 is in a further everted state as the leading edge of the rolling membrane 420 is advanced by a distance x. The inner shaft 450 is in a different position, dislocated by a distance 2x compared to the situation of FIG. 4A. The guidewire 410, which is clamped by the rolling membrane 420 is also clamped by the clamping element 47 such that it is at the same position as in FIG. 4A, as the clamping force of the clamping element 47 exceeds the clamping force of the rolling membrane 420. FIG. 4B thus shows an exemplary scenario for decoupled motion of the guidewire 410 and the rolling membrane 420 reducing the risk of unwanted guidewire 410 advancement.

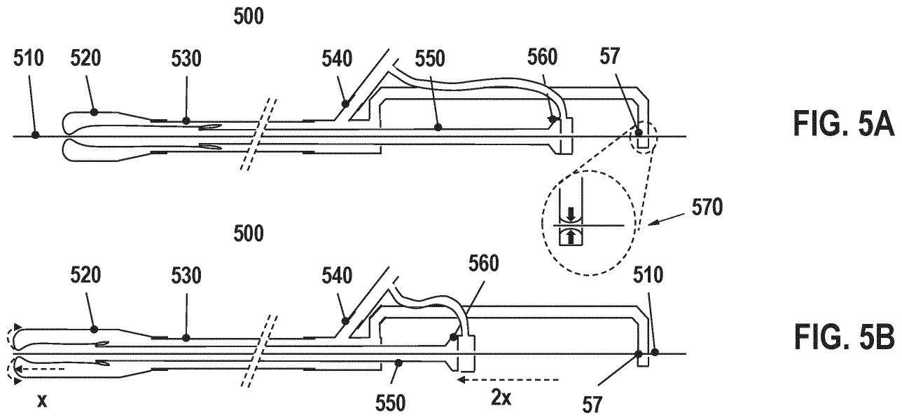

FIG. 5A shows a schematic representation of a rolling membrane catheter 500 with decoupled rolling membrane 520 and guidewire 510 motion via a clamping element 57, e.g. a pressure-controlled clamp 570, and with the rolling membrane 520 in a partly everted state, similarly as outlined with reference to rolling membrane catheter 500 of FIG. 4A. However, catheter 500 additionally comprises a fluid conduit to the inner shaft 550 via the handle 560. Thus, fluid pressure can be provided to the volume between the rolling membrane 520 and the guidewire 510. All three fluid inlets of the outer shaft 530, of the inner shaft 550, and of the pressure-controlled clamp 570 can be connected to the same fluid unit in this exemplary embodiment. The fluid pressure provided to the inner shaft 550 provides a fluid pressure to the volume between the rolling membrane 520 and the guidewire 510, lifting at least a part of the rolling membrane 520 off the guidewire 510. The fluid pressures provided to both sides of the rolling membrane 520 in FIG. 5A are adjusted such that the rolling membrane 520 is in contact with the guidewire 510 only with a small portion at the distal end of the rolling membrane 520. Further, the catheter 500 comprises the second fluid inlet 540 configured to be connected to a fluid unit determining the fluid state of the rolling membrane 520.

Analogously to FIGS. 3B and 4B, FIG. 5B shows the exemplary embodiment of the catheter 500 of FIG. 5A wherein the rolling membrane 520 is in a further everted state as the leading edge of the rolling membrane 520 is advanced by a distance x. The inner shaft 550 is in a different position, dislocated, e.g., via the handle 560, by a distance 2x compared to the situation of FIG. 5A. The guidewire 510, which is clamped by the rolling membrane 520 is clamped by the clamping element 57 and further the friction between the rolling membrane 520 and the guidewire 510 is reduced such that it is at the same position as in FIG. 5A, as the clamping force of the clamping element 57 exceeds the clamping force of the rolling membrane 520. FIG. 5B thus shows an exemplary scenario for decoupled motion of the guidewire 510 and the rolling membrane 520 reducing the risk of unwanted guidewire 510 advancement.

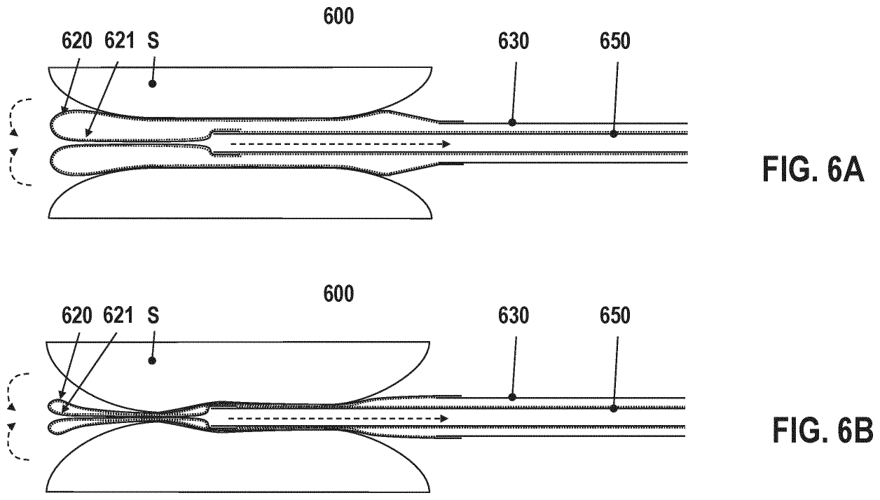

FIG. 6A shows a schematic representation of the retraction of a pressurized rolling membrane (filled with fluid) 620 of a rolling membrane catheter 600 everted through a stenosis S. When the stenosis S is as wide as in FIG. 6A, the inner surfaces of the rolling membrane 620 are not in contact with each other and the rolling membrane 620 can be retracted by retracting the inner shaft 650 in the proximal direction as indicated by the straight dashed arrow. The rounded dashed arrows indicate the rolling motion of the retraction of the rolling membrane 620, essentially the reverse motion of the eversion. The at least one friction-reducing element 621 on the inner side of the rolling membrane does not contribute to the retraction process illustrated in FIG. 6A. The catheter further comprises an outer shaft 630.

FIG. 6B shows a schematic representation of the retraction of a rolling membrane 620 having a friction-reducing element 621 on the inner side everted through a narrow stenosis S. In case the rolling membrane 620 would burst, the stenosis S can collapse and become so narrow that the rolling membrane 620 would be clamped by the stenosis. The inner sides of the rolling membrane 620 provided with the at least one friction-reducing element 621, in FIG. 6B in form of a coating and/or film, shown as dotted lines, are in contact. Without friction-reducing element 621, the friction between the portions of the rolling membrane 620 might be high enough to prevent a controlled retraction of the rolling membrane 620. It might potentially clinch or burst further. The at least one friction-reducing element 621, as present in the exemplary embodiment of FIG. 6B, however, can allow for the controlled retraction of the rolling membrane 620 in a rolling motion by retracting the inner shaft 650 in the proximal direction relative to the outer shaft 630 as indicated by the straight dashed arrow. Like in FIG. 6A, the rounded dashed arrows indicate the rolling motion of the retraction of the rolling membrane 620, essentially the reverse motion of the eversion. Therefore, the at least one friction-reducing element 621 provides a safety measure in case the burst of the rolling membrane 620 cannot be prevented such that the catheter 600 can still be retracted safely.

Both a guidewire advancement and a manual, repeated retraction of the guidewire are dependent on the user (physician). If not taken into account, there is a risk of uncontrolled advancement of the guidewire into distal vascular regions during rolling-out of the rolling membrane and thus a risk of vascular perforation in the patient. Under pressure, the rolling membrane of a rolling membrane catheter is ready for rolling-out and the guidewire is clamped by the rolling membrane. When the inner shaft is advanced and the rolling membrane is roll-outed without the use of a motion compensator, the clamped guidewire is also advanced with twice the speed of the rolling membrane front. The guidewire tip can be pushed far beyond a maximum permissible distal position. This potentially poses a safety risk.

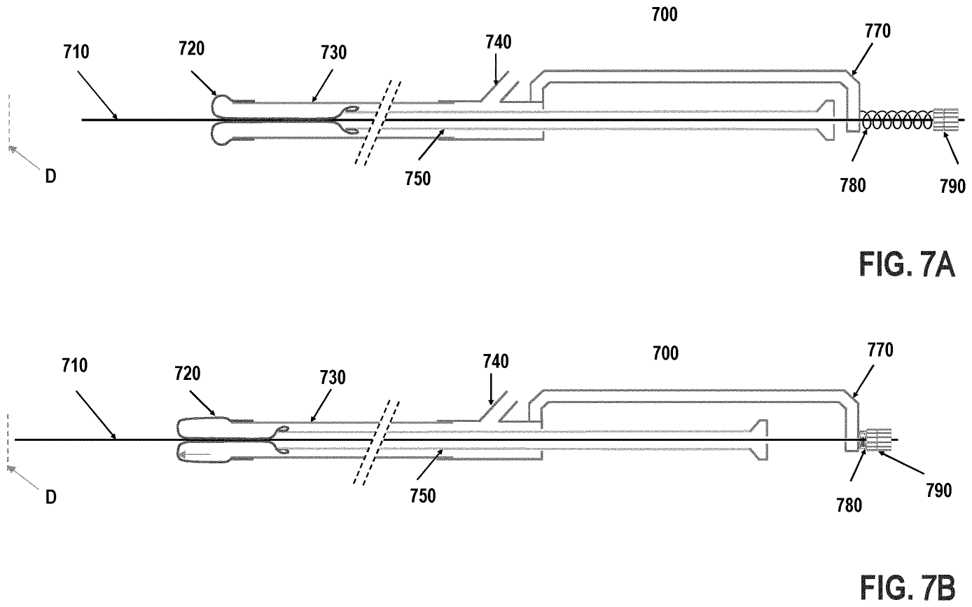

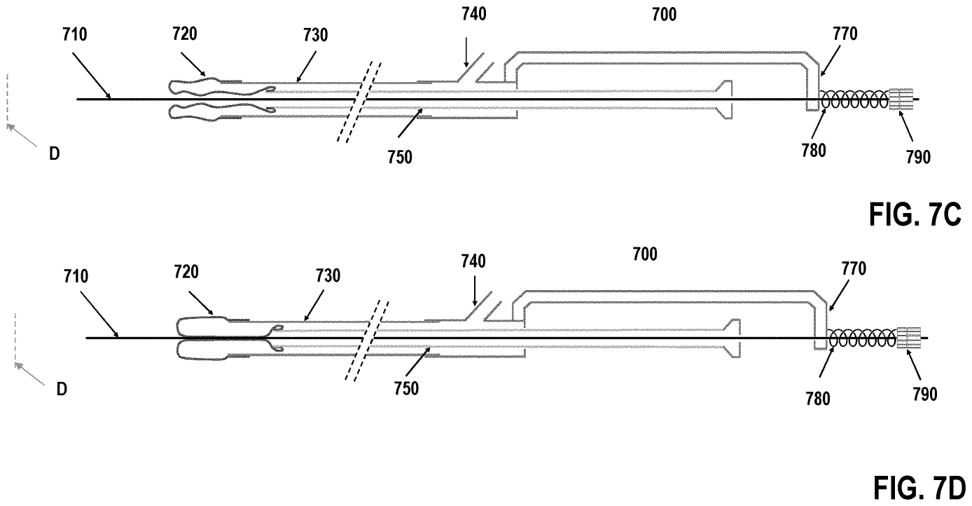

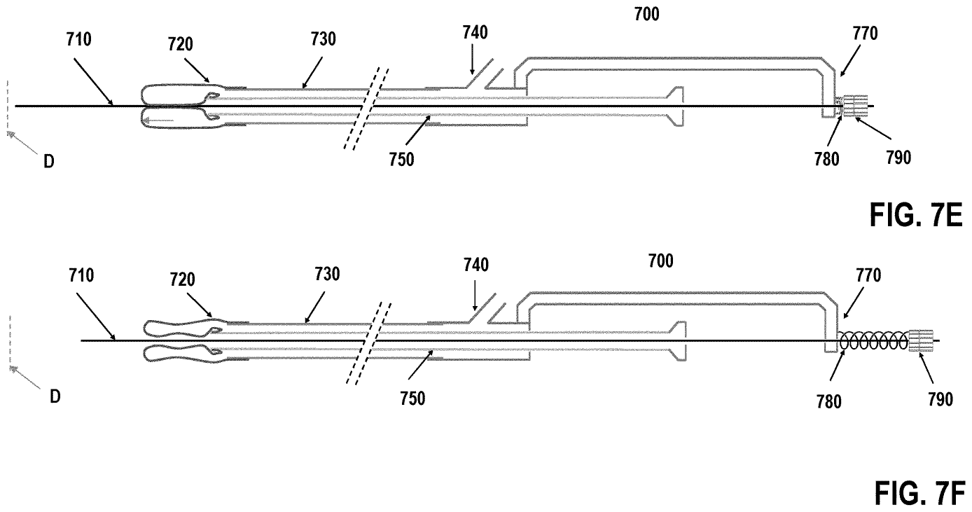

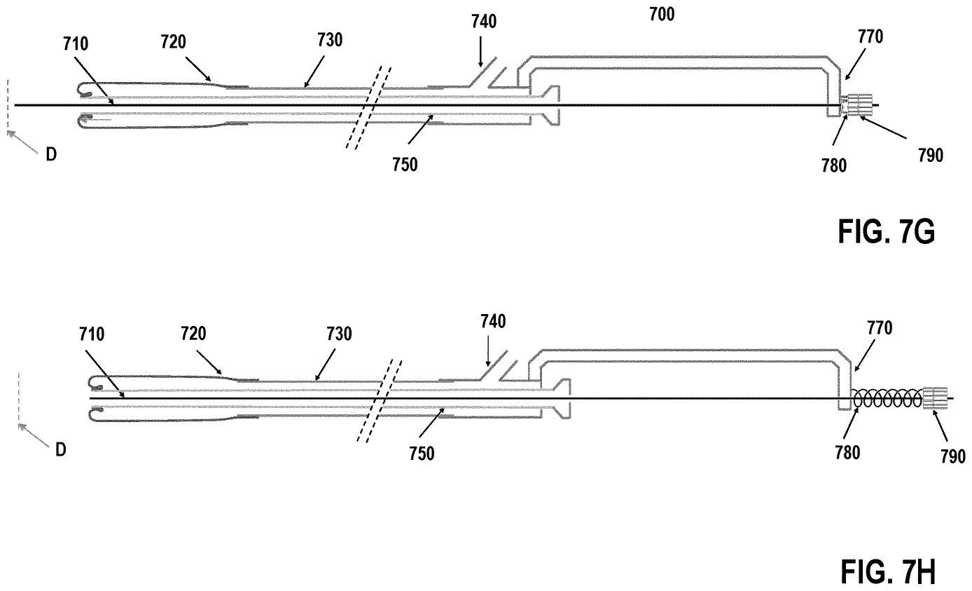

The problem that the rolling membrane and the guidewire movement are coupled can be solved by a rolling membrane shown in FIG. 7A to 7H comprising (at least) one motion compensator 770 configured to limit and compensate the motion of a guidewire advancement. FIG. 7A to 7I show a schematic representation of a rolling membrane catheter 700 with coupled rolling membrane 720 and guidewire 710. The rolling membrane catheter 700 comprises an outer shaft 730 and inner shaft 750. A fluid inlet 740 can be provided and configured to be connected to a fluid unit (not shown) determining the fluid state of the rolling membrane 720 and/or providing a fluid pressure to the rolling membrane volume limited by the rolling membrane 720, the outer shaft 730, and the inner shaft 750. The rolling membrane catheter 700 can further comprises a handle at the proximal end of the inner shaft 750. The (mechanical, cyclic) motion compensator 770 can comprise a torque element 790 and optionally an elastic object 780, preferably a spring. It can be seen that the guidewire 710 is coupled relative to the rolling membrane catheter 700 or the outer shaft 730 by a motion compensator 770 (here comprising a torque element and a coil spring). The motion compensator 770 can be situated at a proximal region of the guidewire 710. D represents the maximal permissible distal position of the guidewire tip. By ensuring a distally limited guidewire advancement in a rolling membrane catheter 700 and thus decoupling this task from the user (physician), the solution significantly increases patient safety in clinical use.

Under pressure (see FIG. 7A), the rolling membrane 720 of a rolling membrane catheter 700 is ready for rolling-out. The guidewire 710 is clamped by the rolling membrane 720. When the rolling membrane is rolled out under pressure (see FIG. 7B), the maximum distal position of the guidewire is limited by the spring path. However, the rolling membrane's rolling path is also limited by the coupling with the guidewire 710. If the pressure in the rolling membrane is temporarily lowered (see FIG. 7C), the guidewire 710 can move freely again. The spring force pushes it back to the initial proximal position. Back under pressure (see FIG. 7D), the rolling membrane 720 of the rolling membrane catheter 700 is ready for further rolling-out. The guidewire 710 is again clamped by the rolling membrane 720. As the rolling membrane 720 continues to roll-out under pressure, the maximum distal position of the guidewire 710 is again limited by the spring path (see FIG. 7E). The rolling distance of the rolling membrane 720 is also limited again by the coupling with the guidewire 710. If the pressure in the rolling membrane 720 is temporarily lowered again (see FIG. 7F), the guidewire 710 can move freely again. It is pushed back into the initial proximal position by the spring force. As the rolling membrane 720 continues to roll-out under pressure (see FIG. 7G), the maximum distal position of the guidewire 710 is limited by the spring path. When the rolling membrane 720 is (almost) completely roll-outed (see FIG. 7H), the guidewire 710 can move freely again. The spring force pushes it back to the initial proximal position.

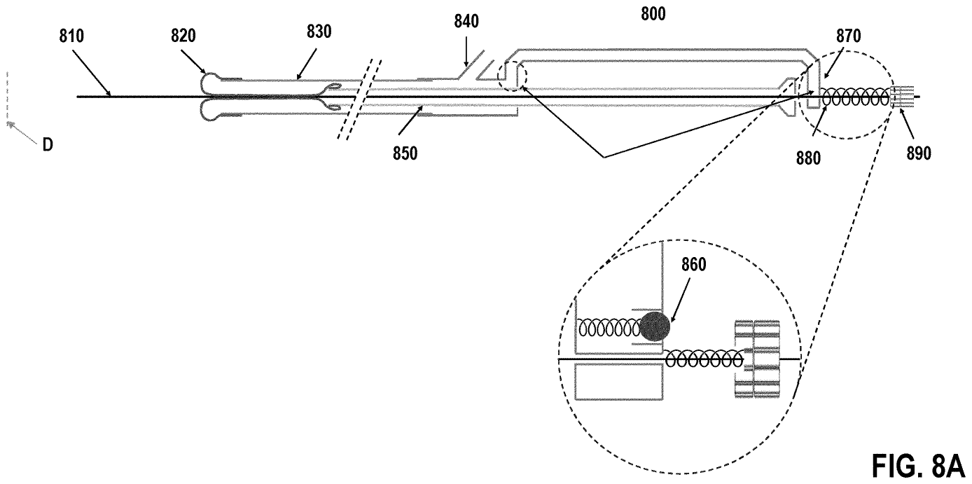

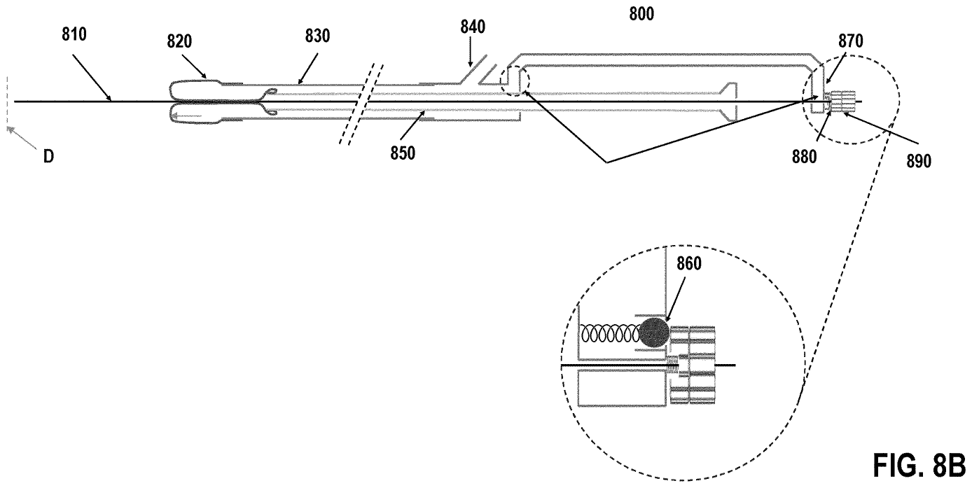

An alternative motion compensator (not shown here) can comprise a torque element but can not comprise an elastic object such as a spring. This allows the physician maximum ease of control over what he wants to do. In such a case the physician can place the guidewire, then advance the rolling membrane catheter on the guidewire until it reaches the stenosis, the guidewire is advanced as distally as is possible and safe. The torque element is now advanced on the guidewire against a bow on the handle and is tightened/locked there. The physician can now pull the guidewire with the torque element back as far as possible without the guidewire losing the path already found. When the rolling membrane is rolled out, the torque element stops the guidewire in the most distal position previously determined by the physician as safe. (The physician must visually monitor that the wire has not taken another branch). Optionally the (mechanical, cyclic) motion compensator of the rolling membrane catheter of FIG. 7A to 7H can be provided with an automatic pressure reduction see (FIGS. 8A/B and 9A/B).

FIGS. 8A and 8B show a rolling membrane catheter 800 comprising a (mechanical, cyclic) motion compensator 870 with an automatic pressure reduction. The rolling membrane catheter 800 further comprises an outer shaft 830 and inner shaft 850. A fluid inlet 840 can be provided and configured to be connected to a fluid unit (not shown) determining the fluid state of the rolling membrane 820 and/or providing a fluid pressure to the rolling membrane volume limited by the rolling membrane 820, the outer shaft 830, and the inner shaft 850. The rolling membrane catheter 800 can further comprises a handle at the proximal end of the inner shaft 850. The (mechanical, cyclic) motion compensator 870 can comprise a torque element 890 and optionally an elastic object 880, preferably a spring. This variant of an automatic pressure reduction uses a ball valve 860. The ball valve seals as long as it does not experience any contact/force from the outside (see FIG. 8A). When the rolling membrane 820 is rolled out under pressure, the maximum distal position of the guidewire 710 is limited by the spring path. The torque element 890 thereby pushes the ball valve 860 inward, causing a small leak and lowering the pressure inside (see FIG. 8A). This reduces the friction between the rolling membrane 820 and the guidewire until the spring force on the torque element 890 is sufficient to pull the guidewire back proximally. At the same time, the ball valve 860 seals and pressure can be built up again in the rolling membrane 820 for the next rolling cycle, where the whole process is repeated.

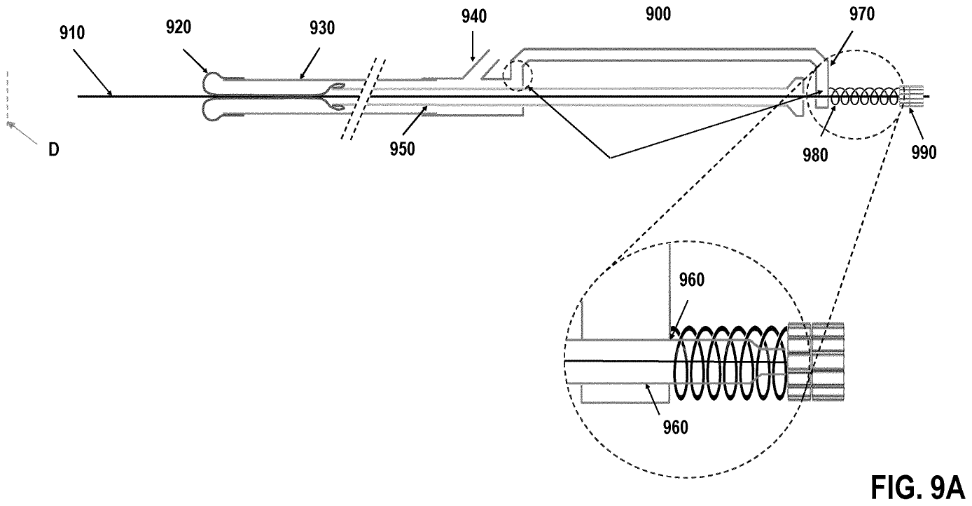

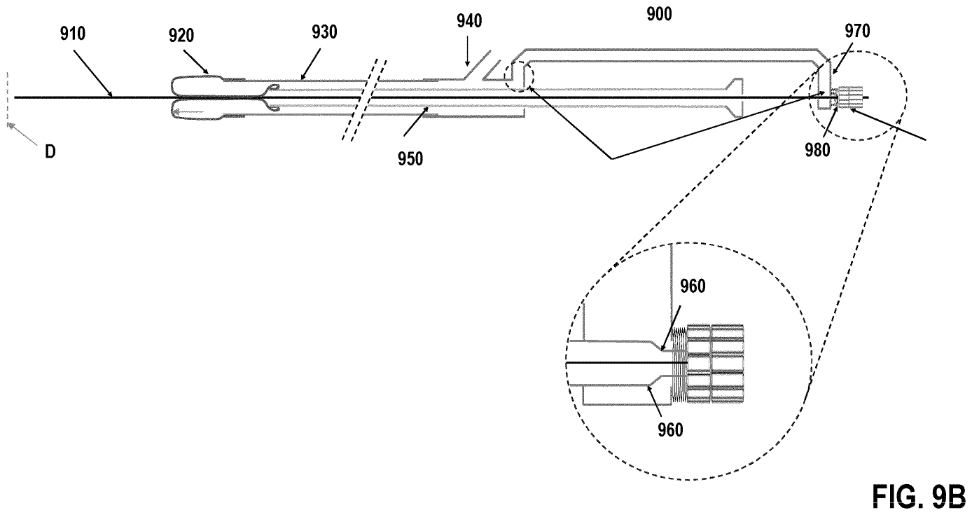

FIGS. 9A and 9B show a rolling membrane catheter comprising a (mechanical, cyclic) motion compensator with an alternative automatic pressure reduction. The rolling membrane catheter 900 further comprises an outer shaft 930 and inner shaft 950. A fluid inlet 940 can be provided and configured to be connected to a fluid unit (not shown) determining the fluid state of the rolling membrane 920 and/or providing a fluid pressure to the rolling membrane volume limited by the rolling membrane 920, the outer shaft 930, and the inner shaft 950. The rolling membrane catheter 900 can further comprises a handle at the proximal end of the inner shaft 950. The (mechanical, cyclic) motion compensator 970 can comprise a torque element 990 and optionally an elastic object 980, preferably a spring. This variant of an automatic pressure reduction uses a tapered envelope as a sliding seal 960. The (hydrophilic) sliding seal 960 seals as long as the non-tapered part of the envelope is in the sliding seal 960 (see FIG. 9A). When the rolling membrane 920 is rolled out under pressure, the maximum distal position of the guidewire 910 is limited by the spring path. As a result, the tapered part of the envelope comes to rest in the area of the sliding seal, creating a small leak and lowering the pressure inside (see FIG. 9b). This reduces the friction between the rolling membrane 920 and the guidewire 910 until the spring force at the torque element 990 is sufficient to pull the guidewire 910 back proximally. At the same time, the sliding seal 960 seals and pressure can once more be built up in the rolling membrane 920 for the next rolling cycle, where the whole process is repeated.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims

1. A catheter comprising:
a rolling membrane;
a guidewire; and
at least one decoupler configured to decouple a motion of the rolling membrane from a motion of the guidewire or at least one motion compensator configured to limit and compensate the motion of a guidewire advancement.

2. The catheter of claim 1, wherein the at least one decoupler comprises a clamping element configured to restrict the movement of the guidewire.

3. The catheter of claim 1, wherein the at least one decoupler comprises at least one friction-reducing element on an outer side of the rolling membrane.

4. The catheter of claim 2, comprising a first fluid inlet configured to be connected to a first fluid unit, wherein the clamping element is configured to restrict the movement of the guidewire when a fluid pressure is provided to the first fluid inlet by the first fluid unit.

5. The catheter of claim 4, comprising a second fluid inlet configured to be connected to the first fluid unit, wherein the first fluid unit is configured to determine a fluid state of the rolling membrane.

6. The catheter of claim 1, wherein the at least one decoupler or the at least one motion compensator is at least in part arranged at a portion of the catheter proximal to the rolling membrane.

7. The catheter of claim 1, wherein the at least one motion compensator comprises a torque element.

8. The catheter of claim 1, wherein the at least one motion compensator comprises a sealing element.

9. The catheter of claim 1, comprising at least one pressure release valve configured to open when a pressure provided to a volume of the rolling membrane exceeds a predetermined threshold and/or at least one friction-reducing element on an inner side of the rolling membrane.

10. The catheter of claim 1, comprising at least one locking structure configured to lock the rolling membrane at least in part based on a fluid state of the catheter.

11. The catheter of claim 10, wherein the at least one locking structure is at least in part connected to at least a portion of an outer shaft of the catheter and/or wherein the at least one locking structure comprises an elastic element configured to change its configuration based at least in part on the fluid state of the catheter.

12. The catheter of claim 11, wherein the at least one locking structure is configured to be in a first configuration locking the rolling membrane when insufficient fluid pressure is provided to the catheter and to be in a second configuration unlocking the rolling membrane when sufficient fluid pressure is provided to the catheter.

13. A system comprising the catheter of claim 5 and at least one fluid unit configured to provide a fluid pressure to at least one of the first fluid inlet and the second fluid inlet.

14. A tubular element comprising a friction-reducing element applied to at least a part of an outer side of the tubular element and/or to at least a part of an inner side of the tubular element for use as the rolling membrane in a rolling membrane catheter of claim 1.

15. A computer program comprising instructions which, when the program is executed, causes the catheter of claim 5 to clamp the guidewire to decouple a motion of the rolling membrane from a motion of the guidewire.

16. The catheter of claim 5, comprising a third fluid inlet configured to be connected to a second fluid unit configured to provide a fluid pressure to a volume between an inner shaft of the rolling membrane and the guidewire.

17. The catheter of claim 7, wherein the motion compensator comprises an elastic object.

18. The catheter of claim 17, wherein the elastic object comprises a spring.

19. The catheter of claim 8, wherein the sealing element comprises a ball valve or a sliding seal.
